(19) United States
(12) Patent Application Publication
(10) Pub. No.: US 2026/0306178 A1
(43) Pub. Date: Oct. 8, 2026

(54) IMAGE GUIDED PROCEDURES

(71) Applicant: DICOM DIRECTOR LLC, NEW HAVEN, CT (US)

(72) Inventors: DAVID PEARLSTONE, NEW HAVEN, CT (US); ANDREW GUAGLIARDO, MOUNT VERNON, NY (US)

(21) Appl. No.: 19/481,691

(22) PCT Filed: May 3, 2024
(86) PCT No.: PCT/US2024/027744

§ 371 Date: Nov. 5, 2025

Related U.S. Application Data
(60) Provisional application No. 63/464,393, filed on May 5, 2023.
(60) Provisional application No. 63/546,072, filed on Oct. 27, 2023.

(51) Int. Cl. A61B 34/10; A61B 34/20; A61B 90/00

(52) U.S. Cl. CPC A61B 34/10; A61B 34/20; A61B 90/37; A61B 2034/105; A61B 2034/2065; A61B 2090/374; A61B 2090/376

(57) ABSTRACT

A system includes at least one image data, a viewing device, and a three-dimensionally printed model (“TDPM”). The at least one image data is generated from an imaging device. The viewing device may have extended reality capability. The at least one image data is optionally configured as a virtual three-dimensional object (“VTDO”). The VTDO is optionally configured as an optical three-dimensional object (“OTDO”). The viewing devices provides a visualization of the VTDO or OTDO. A TDPM is generated from a three-dimensional printing apparatus utilizing the at least one data image file (“image data”), by way of a VTDO or optionally a configuration thereof (e.g., a three-dimensional object, “TDO”). The viewing device harmonizes by co-locating the visualization (e.g., VT-DO or OTDO) with the TDPM.

### CROSS REFERENCE TO RELATED PATENT APPLICATIONS

This application claims the benefit of U.S. Provisional Application No. 63/464,393, filed May 5, 2023, and also claims the benefit of U.S. Provisional Application No. 63/546,072, filed Oct. 27, 2023, the contents of which are hereby incorporated herein by reference in their entirety.

### BACKGROUND

Medical interventions often require invasive procedures. Medical imaging is often used to guide these procedures; these are called image-guided procedures (“IGPs”) and include abscess aspirations, minimally invasive as well as ‘open’ surgical resections, biopsies and many others. Commonly, the medical imaging modalities computer assisted tomography (“CT”) and a magnetic resonance imaging (“MRI” scan (CT and MRI are collectively referred to as “Scans”)scans are evaluated prior to and during a procedure in order to direct the clinician's procedure. The underlying goal is to achieve the desired intervention, such as but not limited to: a resection, biopsy, or aspiration of a structure, such that there is minimal disruption to other tissues.

Invasive medical procedures that require precise approaches rely on imaging to relate the location and relationships between internal structures to the clinician in a meaningful way. Current technologies for this purpose are severely limited, leaving image-guided procedures still within the realm of “guessing” to some extent. The imaging modalities commonly used for image-guided procedures include a Scan. Despite the fact that the images are obtained three-dimensionally, the limitations of previous viewing devices allows for only two-dimensional viewing. These images are visualized as two-dimensional ‘slice’ images and during an image-guided procedure are displayed on a screen physically separate from the patient. This requires the operator to perform the mental process of first re-constructing a three-dimensional image from the series of two-dimensional images and then secondly, mentally superimposing that image onto the patient in order to determine the trajectory of his approach. This process results in inaccuracy and sub-optimal patient outcomes.

Within the last few decades CT and MRI scanners have developed the ability to acquire the images in true three-dimensional fashion. That is to say, during a CT scan, the patient moves horizontally through the sensors in the ‘donut’ at the same time that the sensors are moving circularly within the ‘donut.’ This is distinctly different from previous generations of scanners in which the patient moved horizontally through the ‘donut’ incrementally after each revolution of the sensors. The current generation result is a singe data set that represents a single three-dimensional object as opposed to a series of two-dimensional data sets that can be stacked to represent a three-dimensional object. This single, virtual three-dimensional object (“VTDO”) could only be represented as a ‘planar, 2D image’ on a TV screen because a modality to view three-dimensional images in space did not exist; the volume would then be ‘sliced’ into multiple two-dimensional images that are viewed sequentially. With the advent of extended reality devices (including virtual reality, augmented reality and mixed reality), however, a VTDO can now be viewed in a true three-dimensional viewing format.

Despite the advancement of extended reality devices and VTDO, applications to the medical world requires continued development. For instance, utilizing extended reality devices in training medical personnel fails to tether the information gathered from the scanning technology (e.g., CT or MRI) with the patient. In addition, accuracy remains an issue, both in terms of registration and other technological imperfections, as well as discomfort and/or lack of trust with fully appreciating the capabilities of the technology during a medical procedure by the medical professional and the patient.

Accordingly, it would be desirous to alleviate the aforementioned challenges by way of an improved system, method and apparatus for educational and/or clinical use.

### SUMMARY

A system for image-guided procedures is provided. The system includes in vitro procedures with one or more reference images, preferably one or more three-dimensional reference images. The system includes at least one image data, a viewing device, and a physical model. The viewing device creates a visualization of the at least one image data thereby creating one or more reference images (such as one or more three-dimensional reference images). The physical model is a three-dimensional physical model that is generated from the at least one image data and is printed by a three-dimensional printing apparatus. As such, the physical model is a three-dimensionally printed model. Optionally, the system enables in vivo procedures in addition to in vitro procedures, as described in greater detail below.

One or more controllers are utilized to obtain image data. One or more controllers transfer the at least one image data to a three-dimensional printing apparatus. One or more controllers transfer the at least one image data to a viewing device. The at least one image data is segmented by way of the controller, an interface device or a viewing device. Segmentation is optionally utilized in developing both the physical model and the visualization. In addition to the apparatus described and the generated physical model, the controller utilizes one or more types of algorithms optionally in the form of software to facilitate the aforementioned image data collection, transmission, segmentation, generation of a physical model, and generation of a visualization. Other techniques to refine the image data include registration, volume rendering, and windowing, which can be done by way of a controller, an interface device or a viewing device.

A system includes at least one data image, a viewing device, a virtual three-dimensional object, and a three-dimensionally printed model (“TDPM”). The at least one data image file is generated from an imaging device. The at least one data image file is received by a controller and is thereafter transferred to a viewing device in a suitable file format. The viewing device includes a screen such as a monitor and/or is optionally a wearable device. The viewing device may have extended reality capability. The at least one image data may include a virtual three-dimensional object. The viewing devices provides at least one image generated from an imaging device to a user of the viewing device. The controller transfers the at least one image data in a suitable file format to a three-dimensional printing apparatus. A TDPM is generated from a three-dimensional printing apparatus utilizing a file generated from a medical imaging device. The viewing device harmonizes with the TDPMby co-locating the at least one image data onto the TDPM such that a user is able to manipulate either or both of the TDPM and/or the viewing device. Such manipulation of either or both of the TPDM and/or the viewing device is in real time, where real time is dictated by processing speeds of the controller, the viewing device, refresh rate, and optionally other factors including network (e.g., internet) connection speed, etc. The viewing device provides visualizations commensurate with the TPDM in real time. The viewing device provides a visualization commensurate with externally visible features on the TDPM. The viewing device provides a visualization commensurate with internal features while the TDPM is being interacted with by a user that are obfuscated from the naked eye.

The system includes gathering or receiving the at least one image data that corresponds to, represents, is depicted by and/or can be visualized as a three-dimensional image. The at least one image data [leading to image data that leads to the generation of] the three-dimensional image is of an object, such as but not limited to: a living organism such as an animal or human, including a patient or subject. The patient or subject is one of a human, mammal, animal, reptile, vertebrate, invertebrate, etc. The image data is transmitted to a three-dimensional printing apparatus that optionally utilizes additive manufacturing. A controller assists the three-dimensional printing apparatus. The controller communicates the image data associated with a three-dimensional image (or underlying image data) and communicates the information to the three-dimensional printing apparatus such that a three-dimensional model is precisely created. The three-dimensional imaging apparatus creates and/or prints a three-dimensional printed model of the at least one image data it receives, with the support and/or instructions of the controller. The system further includes software and an interface device. The software receives the at least one image data associated with the three-dimensional image and, by way of extended reality, such as without limitation, augmented reality, provides a visualization of such image data.

The aforementioned will be described in greater detail below, including apparatus', processes, and/or other terminology useful in appreciating the same.

The imaging device is capable of obtaining image data such that a three-dimensional image can be generated. The imaging device is thusly, at minimum, a camera. The imaging device is optionally a medical imaging device. The imaging device is optionally capable of three-dimensional scanning technology such as a CT scanner or an MRI machine. Imaging devices are discussed in greater detail below.

The at least one image data that is obtained from an imaging device such as a CT scanner or MRI machine is called a volume, represents a three-dimensional object and that object can be said to be made up of voxels. A voxel is the representation of a point in space and is the three-dimensional analogue of a pixel; each voxel has four values: x, y and z which describe its location within a Cartesian coordinate system, and p which describes it appearance in some way (color or gray tone).

When viewing a scan in a two-dimensional platform, all the pixels within a given two-dimensional plane are usually depicted; the appearance of each pixel on a screen relative to each other can be varied by a process called ‘windowing’ in which contrast and brightness settings are adjusted to make certain pixel values more evident. The same process can be applied to a volume (the three-dimensional image data from a scan) in a process called ‘volume rendering’.

Volume rendering is distinct from segmentation. In segmentation, specific voxels are selected from the overall volume and those voxels are grouped into a new three-dimensional volume, called a model. By devising specific parameters for selecting which voxels are included in the model, specific anatomic and histologic structures can be isolated from the scan volume and depicted as individual structures themselves. Segmentation software allows a user to select specific structures depicted within a scan by defining algorithms that set appropriate parameters for different structures. The result is the ability to pull specific body parts, such as bones, blood vessels or specific tissues, organs, organ systems, etc., out of the CT scan and view them as individual three-dimensional structures. In certain embodiments, machine learning algorithms are utilized. The output file from a segmentation program represents a new VTDO, depicting a specific set of anatomic structures from the scan.

This output file—the at least one image data-is transferred to a controller with one or more algorithms. The controller and such one or more algorithms include appropriate software systems and hardware. The controller transfers the image data to the three-dimensional printing apparatus to create a physical model such as a three-dimensionally printed model (“TDPM”) of the anatomic structures.

The image data is optionally a VTDO. The VTDO is optionally configured into a three dimensional object (“TDO”) by either the controller or the three-dimensional printing apparatus. The image data, VTDO, or TDO are utilized by the three-dimensional printing apparatus to create the physical model (e.g., TDPM) representative of the information received from the object that underwent the imaging device and/or the scanning technology (e.g., CT or MRI).

The controller transfers the at least one image data into the viewing device to create a visualization. The visualization is an image. The visualization is optionally a three-dimensional image. The visualization is optionally a VTDO. The VTDO is optionally configured into an optical three-dimensional object (“OTDO”) by either the controller or the three-dimensional printing apparatus. The image data, VTDO or OTDO is a manifestation of the information received from the object that underwent the scanning technology (e.g., CT or MRI).

A viewing device is provided and enables a user to see (e.g. visualize) a manifestation of at least one set of image data, the VTDO, or the OTDO. The at least one set of image data is in a suitable file format such that a visualization for the user is provided. The viewing device is optionally an interface device, or other devices as contemplated herein, including monitors (e.g., screens) and extended reality devices.

An interface device provides a visualization of the information (e.g., the at least one image data) to a user, where such visualization is a manifestation of the at least one image data by way of a VTDO or OTDO. The interface device is optionally interactive such that the user is able to input information and/or request information from or with the device. The interface device optionally has a graphical user interface (“GUI”), or is otherwise able to receive inputs from a user and/or provide outputs/information to the user, by way of a touchpad, mouse, stylus, a user's digit, dictation, body movement, etc. Accordingly, an interface device is optionally also a viewing device.

Extended reality (“XR”) is an inclusive term, used to refer to all modalities that involve user immersion to the point of creation of a ‘new reality’. Virtual reality (“VR”), augmented reality (“AR”) and mixed reality (“MR”) are all types of XR. VR hardware devices and software applications are commercially available. VR requires the user to be ‘in a black box’, seeing only the computer-generated reality with no sense of the external (real world) reality. In an AR platform, the user sees the external reality (either through clear lens glasses or external video cameras projecting to the user's eyes) as well as the computer-generated reality. In true AR, however, the computer-generated images are superficially added to the external reality, with no meaningful interaction between the computer images and the real world. AR devices are limited and not widely commercially available. In MR, the hardware device gains spatial awareness and is able to topographically survey and interpret the surrounding environment. With this ability, computer generated images can be placed truly ‘within’ the external reality, providing a dramatic effect. MR requires the capabilities of an AR device and there are a small number of hardware devices becoming available that are specifically designed for MR use.

In an MR environment, computer generated images can recognize objects in the real world; through a process called registration, a computer-generated object can be ‘linked’ to a stationary object in the real world in a specific scale and orientation. The computer-generated image will then remain in the correct position, orientation and scale, regardless of the position of the viewer. In the case of medical scans, models can be created via segmentation from a patient (or subject) scan to depict a specific structure, such as the skeleton. When the model and the patient (or subject) are then viewed through a mixed reality device, the model can be registered to the patient (or subject) to orient the internal structures in the correct anatomical position relative to the actual patient (or subject). This harmonization of the model on the patient (or subject) achieves a level of virtual “x-ray” vision.

A scan as contemplated herein is used to create an image data, or optionally a VTDO. The VTDO is optionally modified into an OTDO in order to generate (e.g., manifest) a visualization through a viewing device, and the VTDO is optionally modified into a three-dimensional object (“TDO”) in order to generate (e.g., create or build) a physical model such as a TDPM of substantially identical anatomic components. The VTDO is generated and represents a subset structure. Optionally, the subset structure is an internal structure of the object that is a subject to the scan that is the target of the procedure. Such an object is, as a non-limiting example, at least a portion of a patient, or subject. The OTDO (or other suitable file and in a suitable file format) is generated and represents additional external structure to allow for accurate registration. In certain embodiments, the OTDO is generated and represents the external structure of the object that is the subject to the scan. In certain embodiments, the OTDO is generated and represents the internal structure of the object that is the subject to the scan. In certain embodiments, the OTDO is generated and represents both the internal and external structure of the object that is the subject to the scan. The OTDO is informative to the investigation of the object and potential procedures related thereto. The physical model such as a TDPM contains the identical structures, but also contains an overlying layer of structures (skin, organ surface, etc.) that obfuscate viewing the targeted internal structures by way of, for instance, the naked eye. The OTDO is visualized through an appropriate XR (e.g., MR or AR) reality headset. Using any number of available registration techniques, the OTDO is harmonized with the physical model such as a TDPM in at least a sufficiently correct orientation and scale. In certain embodiments, the OTDO is harmonized with the physical model such as a TDPM in precisely the correct orientation and scale. The user can perform a mock procedure on the physical model such as a TDPM, using the OTDO as a visual guide.

The term “harmonized” refers to the notion of being able to co-locate the OTDO with the physical model such as a TDPM such that the OTDO, when manipulated by an operator of the viewing device and/or interface (or as in some embodiments, more specifically the XR viewing device or interface associated therewith) and the user views the TDPM through the viewing device, a visualization (e.g., manifestation) of the OTDO in concert with the physical model such as a TDPM at any location on or within the TDPM, is provided. Such harmonization is done with at least one of precise co-location of the OTDO in concert with the TDPM, and accurate co-location of the OTDO on the TDPM. In certain embodiments, the precision of the co-location of the OTDO in concert with the TDPM is at least 80%, or at least 90%. In certain embodiments, the accuracy of the co-location of the OTDO in concert with the TDPM is at least 80%, or at least 90%. In such fashion, the OTDO is visualized as an overlay and/or superimposition onto the physical model such as a TDPM such that the operator of the viewing device is able to manipulate the viewing device and/or interface in order to see a specific portion of the physical model such as a TDPM in concert with the OTDO, where such specific portion of the physical model such as a TDPM that may or may not be viewable to the naked eye.

A system for visually guided in-vitro procedures is provided. The system includes one or more image data having a file format. The one or more image data includes a first tissue or organ. The one or more image data includes a second tissue or organ. The first tissue or organ is different from the second tissue or organ.

The system includes one or more physical models. The physical model is optionally a TDPM. The TPDM is optionally configured from a VTDO file. The VTDO file is optionally configured from the one or more image data. The TPDM is optionally configured from a TDO file. The TDO file is optionally configured from the VTDO file.

The one or more image data provides a VTDO including the first tissue or organ. The one or more image data provides a VTDO including the second tissue or organ. The one or more image data provides a VTDO including the first tissue or organ and the second tissue or organ. The VTDO provides a TDO including the first tissue or organ. VTDO provides a TDO including the second tissue or organ. The VTDO provides a TDO including the first tissue or organ and the second tissue or organ.

The VTDO is utilized to generate an OTDO. The VTDO is configured into an OTDO. The VTDO is viewable on at least one or more viewing devices. The OTDO is viewable on at least one or more viewing devices.

The one or more image data is utilized to generate a TDPM. The VTDO is optionally utilized to generate a TDPM. The TDO is optionally utilized to generate a TDPM. The TDPM optionally includes a first tissue or organ. The TDPM optionally includes a second tissue or organ.

The at least one viewing device is optionally an XR device. The XR device is optionally an AR device. The XR device is optionally an MR device. The OTDO is viewed through the XR device, and/or optionally through an AR device, and/or optionally through an MR device.

The OTDO is viewed through the XR device generating a visualization that is harmonized with the TDPM. The TPDM and visualization are harmonized such that the visualization of the first tissue or organ is synchronized through the XR device with the TDPM. The TPDM and visualization are harmonized such that the visualization of the second tissue or organ is synchronized through the XR device with the TDPM. The TPDM and visualization are harmonized such that the visualization of the first tissue or organ and the second tissue or organ is synchronized through the XR device with the TDPM.

Optionally, the one or more viewing devices further comprises a screen.

Optionally, the visualization is harmonized with the TDPM by a command inputted to an input device. Optionally, the input device is an XR device. Optionally, the harmonization occurs in real-time. Optionally, a user of the XR device is able to synchronize the OTDO and the TDPM as the user interacts with at least the TDPM in real time.

Optionally, the visualization is harmonized with the TDPM by a command inputted to an input device. Optionally, the input device is an XR device. Optionally, the harmonization occurs in real-time.

Optionally, a user of the XR device is able to harmonize the OTDO and the TDPM as the user interacts with both the TDPM and the OTDO in real time.

Optionally, the system includes an object. The object is optionally a patient or a subject. The patient or subject is optionally subject to a scanning device. The scanning device is optionally a MID.

Optionally, the at least one image data corresponds to at least a portion of the object. Optionally, the at least one image data corresponds to at least a portion of a patient or subject. Optionally, the at least one image data is a VTDO, a TDO, or an OTDO, and corresponds to at least a portion of the object, or optionally the patient, or optionally the subject.

Optionally, the visualization corresponds to the VTDO or the OTDO, where the VTDO or the OTDO corresponds to the at least one image data of the object or patient or subject. Optionally, the visualization is harmonized with the TDPM corresponding to the portion of the object, or the patient or the subject.

Optionally, the first tissue or organ is an external tissue or organ. Optionally, the first tissue or organ is viewable (e.g., to the naked eye). Optionally, the second tissue or organ is an internal tissue or organ. Optionally, the second tissue or organ is obfuscated (e.g., from the naked eye).

Optionally, the system includes a third tissue or organ. The third tissue or organ is optionally different from the first tissue or organ, and/or is different from the second tissue or organ.

Optionally, the one or more image data, and/or the VTDO, and/or the OTDO, undergo segmentation. Optionally, the one or more image data, and/or the VTDO, and/or the OTDO, undergo registration. Optionally, the one or more image data, and/or the VTDO, and/or the OTDO, undergo segmentation and registration.

Optionally, the TDPM includes a first material. Optionally, the TDPM includes a second material. The first material is optionally different from the second material. The first material and the second material are optionally different colors. The first material and the second material optionally have different material properties. The first material and the second material are optionally different materials.

A method for a visually guided procedure is provided. The method includes obtaining at least one image data (e.g., a VTDO). The VTDO includes a first tissue or organ. The VTDO includes a second tissue or organ.

The method includes creating a TDO from the VTDO.

The method includes creating a TDPM from the TDO. The TDPM is configured to have a first tissue or organ. The TDPM is configured to have a second tissue or organ.

Optionally, the method includes creating the TDPM such that the first tissue or organ is different from the second tissue or organ.

The method includes manifesting an OTDO from the VTDO through at least one of an interface device and a viewing device. The interface device is optionally an XR device. The viewing device is optionally an XR device. The XR device is optionally an AR device. The XR device is optionally an MIR device.

Optionally, the method includes registering the OTDO.

Optionally, the method includes registering the visualization through the viewing device.

Optionally, the method includes harmonizing the visualization with the physical model when viewed through the viewing device.

Optionally, the method includes harmonizing the OTDO with the physical model when visualized through a viewing device.

Optionally, the method includes registering the OTDO in order to harmonize the OTDO with the physical model when visualized through the viewing device.

Optionally, the method includes a visualization of the first tissue or organ. Optionally, the method includes a visualization of a second tissue. The visualization of the first tissue or organ is through the viewing device. The visualization of the second tissue or organ is through the viewing device.

Optionally, the method includes synchronizing the visualization through the viewing device with the TDPM such that a user is able to interact virtually with the OTDO and physically with the TDPM.

Optionally, the method includes a user interacting with the TDPM through the visualization.

Optionally, the method includes a user interacting physically with the TDPM. The user physically interacts with the TDPM through tactile or touching.

Optionally, the viewing device further includes a screen.

Optionally, the method further includes a second user. The second user interacts with a second viewing device. The second viewing device includes a screen. The second viewing device includes a second XR device. The second XR device is one of an AR device and an MR device.

The method optionally includes creating a VTDO from at least one image data. The method optionally includes creating at least one image data is created from an imaging device and/or a scanning device. The scanning device is optionally an MID.

The method optionally includes scanning an object with the scanning device. The object is optionally a patient or subject.

The method optionally includes obtaining at least one image data from the scanning device.

The method optionally includes transmitting the at least one image data from the scanning device to a controller. The at least one image data is optionally a VTDO.

The method optionally includes transmitting the at least one image data from the controller to a three-dimensional printing apparatus. The at least one image data is optionally a VTDO.

The method optionally includes configuring the at least one image data into a VTDO. The method optionally includes configuring the VTDO into a TDO. The method optionally includes generating a physical model from the TDO. The physical model is optionally a TDPM.

The method optionally includes transmitting the at least one image data from the controller to a viewing device. The at least one image data is optionally a VTDO. The viewing device is optionally a screen. The viewing device is optionally a wearable device. The viewing device is optionally an XR device, such as an AR device, and/or an MR device.

The method optionally includes configuring the VTDO into an OTDO utilizing the XR device.

The foregoing features and elements may be combined in various combinations without exclusivity, unless expressly indicated otherwise. For example, aspects and/or embodiments of the present disclosure may include any one or more of the individual features or elements disclosed above and/or below alone or in any combination thereof. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 depicts an embodiment according to the present disclosure.

FIG. 2 depicts an embodiment according to the present disclosure.

FIG. 3 depicts an embodiment of the TDPM according to the present disclosure.

FIG. 4 depicts an embodiment of the visualization according to the present disclosure.

FIG. 5 depicts an embodiment of the visualization with the TDPM utilizing a viewing device according to the present disclosure.

FIGS. 6A-6C depict an embodiment of the visualization with an object utilizing a viewing device according to the present disclosure.

FIGS. 7A-7B depicts embodiments of the interface according to the present disclosure.

FIGS. 8A-8B depicts embodiments of the interface according to the present disclosure.

### DETAILED DESCRIPTION

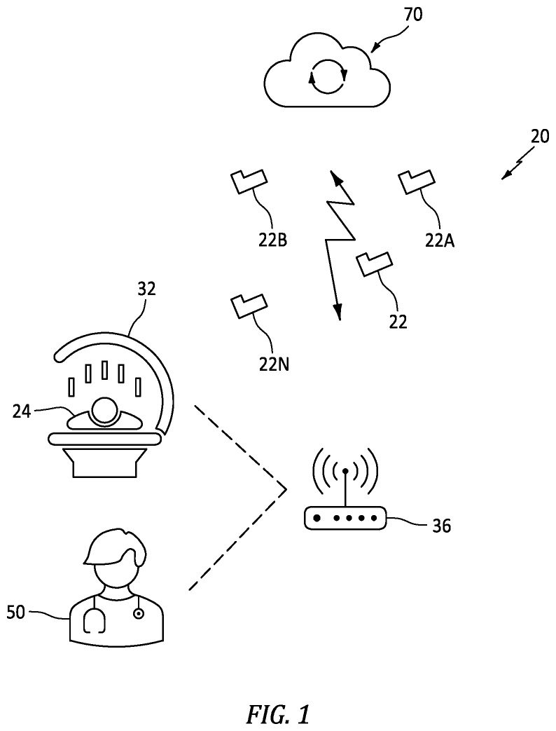

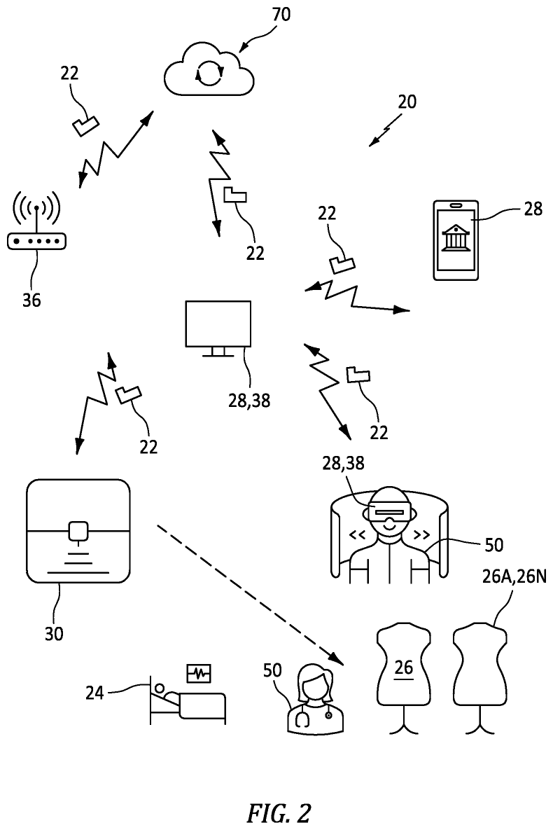

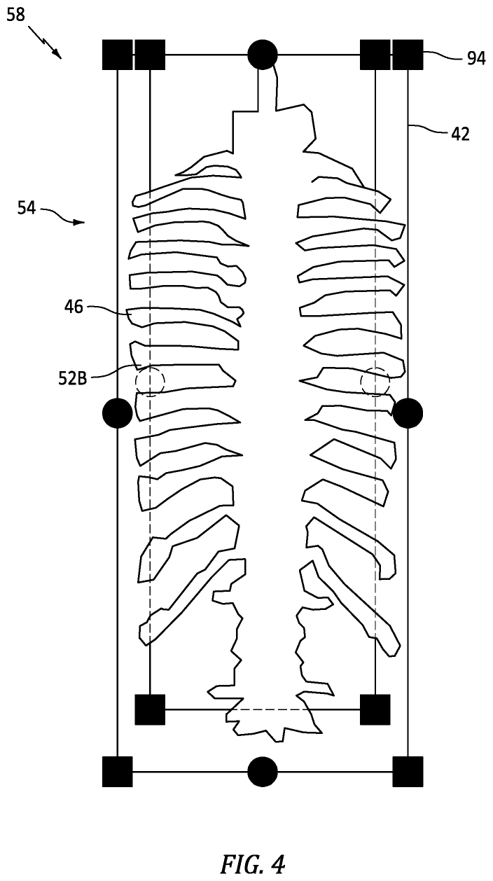

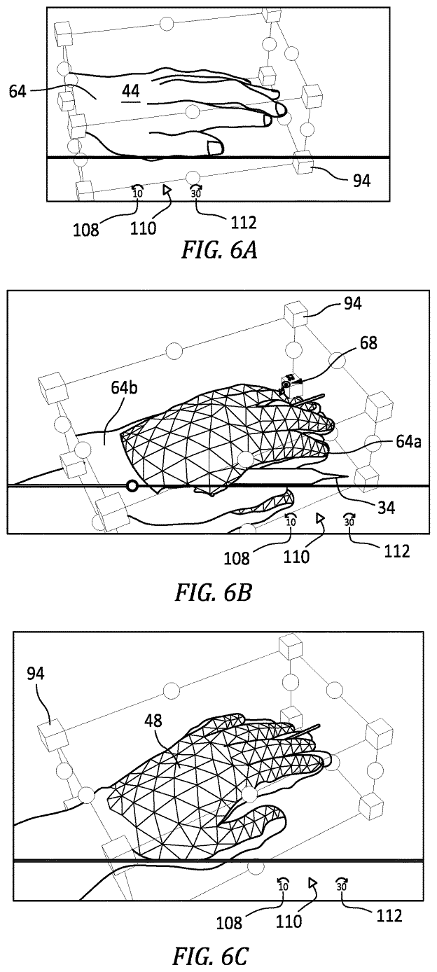

Referring generally to exemplary FIGS. 1-6C, the present disclosure regards a system for an image guided procedure 20. The image guided procedure 20 includes at least one image data 22 of an object 24 (e.g., an object, a patient or subject), a physical model 26, and at least one viewing device 28. Optionally, a three-dimensional printing apparatus 30 is provided. Optionally, an imaging device 32 or other device capable of (i) compiling information to generate image data that can be regressed into an image, or (ii) capturing an image, is provided. Optionally, one or more medical instruments 34 is(are) provided, such as a probe, a tool, etc. (see FIG. 6B). Optionally, one or more controllers 36 are provided. In addition to the aforementioned, several methods are provided, including interactive steps with the interface device 38 that is optionally a viewing device 28, further optionally including segmentation 40 and/or registration 42 (see FIG. 4). These components and methods will be described in greater but non-limiting detail below, including optional components.

Image data 22 related to an object 24 (e.g., a patient or subject, etc.), includes data regarding external features 44 such as external anatomical features 44 (e.g., the epidermis, or other features, including superficial features viewable to the naked eye) as well as internal features 46 such as anatomical internal features 46 (e.g., the lungs) that are obfuscated by external features 44 such as external anatomical features 44. For simplicity, any object 24 referred to herein, such as without limitation, a patient or subject, will generally be identified by reference numeral 24. For further simplicity, an external feature 44 is often an external anatomical feature 44, and as such, any external feature may be referred to by reference numeral 44. Similarly, an internal feature 46 is often an internal anatomical feature 46, and as such, any internal feature may be referred to by reference numeral 46. Image data 22 of the patient or subject 24 includes at least a portion or region of the patient or subject 24, such as the axial region, the appendicular region, the head, neck, torso, pelvis, lower extremities, upper extremities, etc. The image data 22 can include a coronal, sagittal or cross-sectional portion or region of the patient or subject 24. The image data 22 of the patient or subject 24 includes specific tissues and/or tissue or organ systems such as without limitation: the lymphatic system, circulatory system, respiratory system, endocrine system, nervous system, reproductive system, digestive system, urinary system, integumentary system, muscular system, skeletal system, etc. Optionally, the image data 22 includes the entirety of the object (e.g., the entire body of the patient or subject 24). Optionally, image data 22 is a VTDO 22, whereby the image data 22 regards any and all tissues/organs within a given portion of the body of the patient or subject 24.

In certain embodiments, a second set of image data 22A is provided. This second set of image data 22A regards the patient or subject 24 in a second condition that is different than the patient or subject 24 in a first condition captured in the first image data 22. Alternatively, the second set of image data 22A regards a different patient or subject 24 than the first image data 22 of the first patient or subject 24. Alternatively, the second set of image data 22A regards a medical professional or trainee who is performing a related medical procedure. In such alternative embodiment, the second set of image data 22A regarding the medical personnel includes the region(s) of the medical professional's or trainee's body 48 that will interact in the medical procedure, such as the medical person's appendage(s), arm(s), hand(s), digit(s), (i.e., finger(s)), etc. For simplicity, any image data 22 referred to herein, such as without limitation, a first set of image data, a second set of image data, etc. may be identified simply by reference numeral 22.

In certain embodiments, image data 22 includes a first set of image data 22, a second set of image data 22A, and a third set of image data 22B. Optionally, a fourth set of image data 22 is provided, and further optionally, an nth set of image data 22N. In such embodiments, the second set of image data 22A, the third set of image data 22B, and the fourth set of image data 22 are from one or more of the patient or subject 24 in a second state, third state, fourth state, a benchmarking patient or subject 24 that is different than the patient or subject 24, and/or that of a medical professional or trainee 48. It follows that an nth set of image data 22N may be provided, whereby such nth set of image data 22N regards one or more of an nth state of the object 24 such as a patient or subject, a second state of a benchmarking patient or subject 24, an nth benchmarking patient or subject 24, an nth medical professional or trainee 48, etc.

The at least one image data 22 can be in a variety of electronic file formats, such as .OBJ, .MTL (e.g., file format for color and shading generally utilized with a .OBJ file), STL, .3MF, .X3D, .WLR, etc. In certain embodiments and as discussed in further detail below, the image data 22 utilized is from the same root file to generate the physical model 26 and as utilized by the viewing device 28, albeit the root file (e.g., VTDO 22) is configured into a TDO for the physical model 26 and an OTDO for the visualization 58.

The physical model 26 is created from the at least one image data 22. The physical model 26 is a tangible representation of the patient/subject 24 or as otherwise described herein (e.g., a benchmarking patient or subject 24), regarding external anatomical features 44 (e.g., the epidermis) as well as internal anatomical features 46 (e.g., the lungs). The physical model 26 of the patient or subject 24 includes at least a portion or region of the patient or subject 24, such as the axial region, the appendicular region, the head, neck, torso, pelvis, lower extremities, upper extremities, etc. The physical model 26 can include a cross-sectional or sagittal portion or region of the patient or subject 24. The physical model 26 of the patient or subject 24 includes specific tissues and/or tissue or organ systems such as without limitation: the lymphatic system, circulatory system, respiratory system, endocrine system, nervous system, reproductive system, digestive system, urinary system, integumentary system, muscular system, skeletal system, etc. Optionally, the physical model 26 includes the entire body of the patient or subject. Preferably, physical model 26 is a TDPM, whereby the physical model 26 regards any and all tissues/organs within a given portion of the body of the patient or subject 24. For the purposes of the present disclosure, any reference to “physical model” 26 includes TDPM 26 and other physical models 26. For simplicity, any physical model 26 is referred to by reference numeral 26.

In some embodiments, a second physical model 26A is provided. This second physical model 26A information regards the patient or subject 24 in a second condition that is different than the patient or subject 24 in a first condition captured in the first physical model 26. Alternatively, the second physical model 26A regards a different patient or subject 24 than the first physical model 26 of the first patient or subject 24. It follows that an nth physical model 26N may be provided, whereby such nth physical model 26N regards one or more of an nth state of the patient or subject 24, a second state of a benchmarking patient or subject 24, an nth benchmarking patient or subject 24, etc. Any such physical model 26 according to the present disclosure is optionally a TDPM 26.

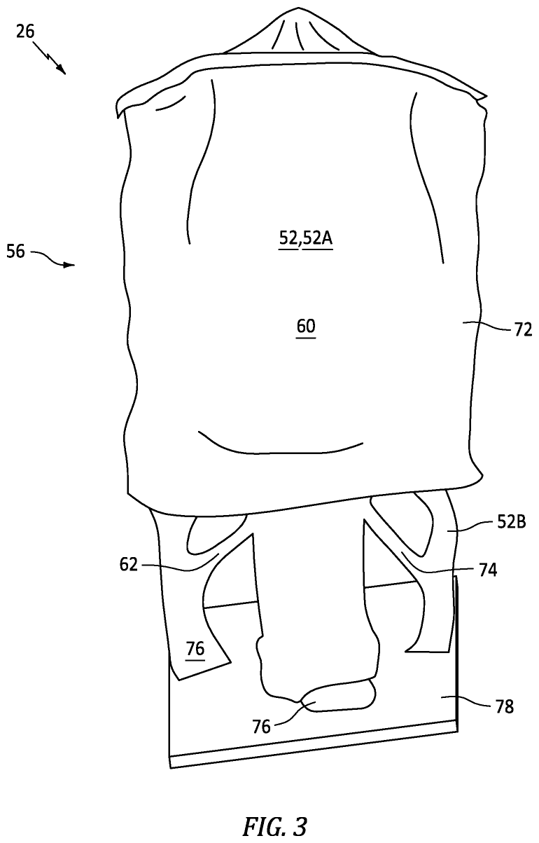

By way of a non-limiting example and as shown in FIG. 3, a representation of a TDPM 26 created from the same VTDO 22 as the augmented reality image depicted in FIG. 4, such as an OTDO. The TDPM 26 is optionally created from a TDO 22, configured from the VTDO 22.

The viewing device 28 is used by a viewer 50 such as a user or operator, or in further non-limiting examples, such as a medical professional or trainee, a patient, etc., to see (e.g. view, visualize) a representation of the one or more image data 22. For the purposes of the present disclosure, viewer, user, and operator are used interchangeably and all are referred to by reference numeral 50. The viewing device 28 provides a visualization 58 of the at least one image data 22 to the viewer 50 without completely obfuscating the viewer's 50 ability to see by way of the naked eye (or as assisted by glasses, contacts, etc.). The viewer 50 is optionally able to interact with the one or more image data 22 (that is optionally configured as a VTDO or further optionally as an OTDO), such as by way of an interface device 38 such as a graphical user interface (GUI), a keyboard, mouse, stylus, digit (i.e., a finger), dictation, body movement, etc. For simplicity, any interface device 38 may be referred to by reference numeral 38. The viewer 50 is optionally able to set or define parameters with respect to viewing the one or more image data 22 (that is optionally configured as a VTDO or further optionally as an OTDO). The viewing device 28 includes a screen. The viewing device 28 is optionally attached to a controller 36 (as discussed in greater detail below) or is a standalone screen or a plurality (e.g., series, an array, etc.) of screens. The one or more screens may be generally planar or may be arcuate, having a curvature such that one or more edges of the screen is in a second plane parallel to a first plane in which a center portion of the screen is. The viewing device 28 optionally includes a projection by way of a projector and a generally planar surface such as a projection screen. The viewing device 28 is optionally held by, worn by, or attached to a user 50. In certain embodiments, the viewing device 28 is an XR device 28a, such as a VR, MR, and/or AR device. In such embodiments, the viewing device 28 is worn about the user's 50 head, such as over the user's 50 eyes. In such embodiments, the viewing device is, including without limitation: HOLOLENS, OCULUS PRO, APPLE VISION PRO, MAGIC LEAP ONE, GOOGLE GLASS, MOVERIO BT300, NVIS, BROTHER AIRSCOUTER, WD-100, ARYZON, METAVISION, PICOLINKER,, and/or VUZIX M300. For simplicity, any viewing device 28 may be referred to by reference numeral 28.

By way of a non-limiting example and as shown in FIG. 4, a representation of a visualization such as an OTDO 22 represents the boney thorax of a human subject. The OTDO 22 was created by segmentation 40 of the at least one image data 22 generated by a CT scanner 32 depicting the thorax of a human subject and is visualized with INTRAVISION XR software from DICOM Director, LLC, and viewed as a OTDO 22 through the MICROSOFT HOLOLENS 2A viewing device 28.

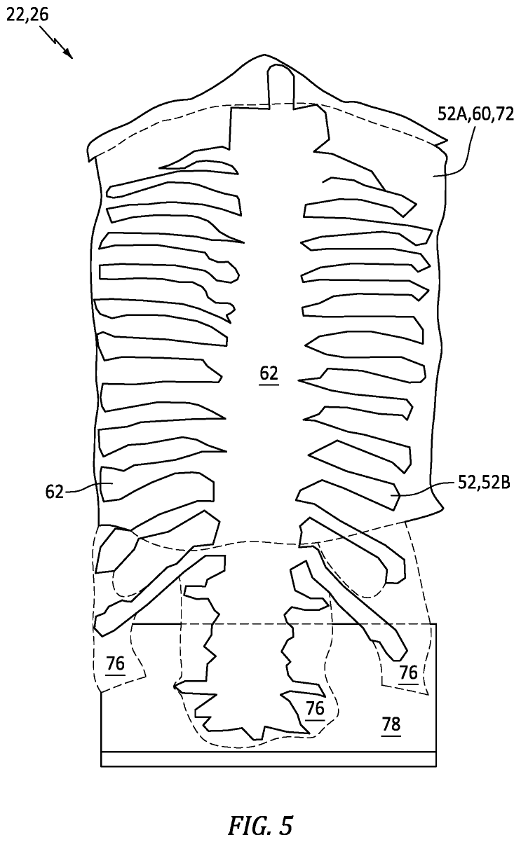

Referring now to exemplary FIG. 5, the at least one image data 22 configured into a VTDO and or further optionally as an OTDO is viewable on the viewing device 28 and by way of software or user inputs, is registered to the physical model created from the same image data. By way of software or user inputs, additional modifications can be made, including segmentation 40, volume rendering, windowing, etc.

The internal features 46 visualized on the viewing device 28 can be selected as contemplated above such that a certain tissue and/or portion of an organ system 52 is shown while tissues and/or portions of organ systems 52a are hidden. Similarly, a physical model 26 can be created by selectively including only certain tissue and/or portion of an organ system 52. By way of non-limiting example, FIG. 4 shows bones 52 (which are at least one of the internal anatomical features 46) covered by the dermis 52 (which is at least one of the external anatomical features 44). By way of further non-limiting example, FIG. 6A shows soft tissue 52. Segmentation 40 methods and programs are known and those generally available are considered for purposes of the present disclosure.

Through the viewing device 28, one or more respective layers 54 of the visualization 58 correlate to one or more respective layers 56 on or within the physical model 26. Said differently, the visualization 58 through the viewing device 28 is harmonized with the TDPM 26. By way of example, the external anatomy 60 of the physical model 26 correlates to the external anatomical features 44 shown through the viewing device 28. By way of a second example, as the user 50 interacts with the physical model 26 by way of internal anatomical features 46, the viewing device 28 shows correlating internal features 46. The external features 44 and/or internal features 46 visualized on the viewing device 28 can be selected as contemplated above such that a certain tissue and/or portion of an organ system 52 is shown while others are hidden. Similarly, a physical model 26 can be created by selectively including only certain tissue and/or portion of an organ system 52.

In certain embodiments, the external anatomy 60 of the physical model 26 is the same as the external anatomical features 44 in the visualization 58 as seen by the user 50 through the viewing device 28. In such embodiments, the one or more data files 22 optionally configured thereafter as a VTDO and/or further optionally as a TDO generated the physical model 26, and also optionally configured as a VTDO and/or further optionally as an OTDO for the visualization 58 by the user 50 through the viewing device 28. In certain embodiments, the internal anatomy 62 of the physical model 26 is the same as the internal anatomical features 46 seen by the user 50 through the viewing device 28. In such embodiments, the one or more image data 22 generated both the physical model 26 and also what is seen in the visualization 58 by the user 50 through the viewing device 28.

In certain embodiments, a first image data set 22 corresponds to a first patient or subject 24 and a second image data set 22 corresponds to a second patient or subject 24. Alternatively, a first image data set 22 corresponds to a patient or subject 24 in a first condition and a second image data set 22A corresponds to a patient or subject 24 in a second condition. In either embodiment, the physical model 26 is generated by way of the three-dimensional printing apparatus from the first image data set 22. The visualization 58 as seen by a user 50 of the viewing device 28 is generated from the second image data set 22A. The user 50 of the viewing device 28, by way of registration 42, is able to generally co-locate the visualization 58 to the physical model such that one or more respective layers 54 of the visualization 58 generally correspond to one or more layers 56 of the physical model 26.

In further embodiments, a physical model 26 and viewing device 28 are used in concert with the object (e.g., patient or subject) 24. A first set of image data 22 is associated with a physical model 26. The viewing device 28 provides a visualization 58 of the first set of image data 22. The visualization 58 via the viewing device 28 or interface device 38 is registered to at least the physical model 26. Optionally, the visualization 58 is also registered to the patient or subject 24. At least a second set of image data 22A is optionally provided and relates to one or more of the following: the object (e.g., patient or subject) 24 in a second state that is different than the first state that led to the first set of image data 22A, or a control patient or subject 24. In one such embodiment, the at least second set of image data 22A regards the object (e.g., patient or subject) 24 (or control patient or subject 24) in a second state that is different than the first state that led to the first set of image data 22, whereby the first state and the second state regard extents or extreme conditions of at least a portion of the object (e.g., patient or subject) 24 (or control patient or subject 24) during the procedure. For instance, if the procedure regards the respiratory system, the first state is optionally when the lungs are completely deflated (e.g., are not filled with a fluid such as air), and the second state is optionally when the lungs are completely Inflated (e.g., filled with a fluid such as air). Similar to the aforementioned exemplary embodiments, the internal features 46 in the visualization 58 by way of the viewing device 28 can be selected as contemplated above such that a certain tissue and/or portion of an organ system 52 is shown while others are hidden. Similarly, a physical model 26 can be created by selectively including only certain tissue and/or portion of an organ system 52.

As exemplified in FIGS. 6A-6C, in embodiments utilizing XR technologies such as MR, objects 24 subject to a visualization 58 by way of the XR viewing device 28 (e.g. MR viewing device 28) can be seen as-is. For instance, a portion of the user 50 such as an arm or hand may be viewable in the visualization 58, and similarly, implements or objects 24 are also viewable (e.g., tables, medical instruments 34, etc.) in the visualization 58. In some embodiments, a portion of the user 50 is a mapped portion 64 such that the interface device 38 and/or XR viewing device 28 will respond to and/or perform specific tasks upon specific body movement of the mapped portion 64. For instance, when using HOLOLENS®, one or both of a user's 50 wrists and one or both of a user's index fingers are mapped portions 64. Upon a user 50 putting his/her index finger 64a on one hand on his/her wrist (e.g., the upper side or the lower side of the wrist) 64b adjacent the other hand will prompt a menu and/or command 66 to the viewing device 28 and/or interface device 38. In certain embodiments, once a user 50 putting his/her index finger 64a on one hand on his/her wrist 64b adjacent the other hand and a menu 66 is prompted, the viewing device 28 and/or the interface device 38 will allow the user 50 to enter a further command 66 manually or virtually. In other embodiments, a button or a menu 66 is viewable and/or can be interacted with on the viewing device 28 and/or interface device 38. The user 50 is able to, by virtue of the user's finger 64a being a mapped portion 64, is able to virtually touch the button 66 (at least as seen on the visualization 58 created by the viewing device 28) and thereby make a selection or other command in accordance with the button 66. Such examples of portions of a user's 50 body that are mapped portions 64 are exemplary and non-limiting.

In further embodiments utilizing XR technologies such as MR, one or more trackers 68 can be placed on a user 50 or object 24 to enable the XR device 28 to identify the location of such portion of a user 50 and/or portion of an object 24 having the one or more trackers 68. Utilization of one or more trackers 68 enables the viewing device 28 to further harmonize the visualization 58 (e.g., VTDO or OTDO 22) and TDPM 26 with what is being mapped by the one or more trackers 68 (e.g.'s: a portion of a user 50 such as a hand, a portion of an object 24, or further items such as a medica′ instrument 34, a portion of a prosthesis, combinations thereof, etc.).

In some embodiments, the one or more trackers 68 are configured on the user 50 and/or object 24 such that as the user 50 and/or object 24 and/or medical instrument 34 engage with (e.g., interact with, move toward to be proximal to, penetrate, manipulate, etc.) a respective layer 56 (e.g., going from the external anatomy 60 to the internal anatomy 62) of the TDPM 26, the visualization 58, likewise proceeds to the correlating respective layer 54 (e.g., going from external features 44 to internal features 46). In some embodiments, the one or more trackers 68 are configured on the user 50 and/or object 24 and/or medical instrument 34 such that when, through the viewing device 28 and/or the interface device 38, while the user 50 and/or the object 24 including the one or more trackers 68 engages with (e.g., interacts with, moves towards to be proximal to, penetrate, manipulate, etc.) a respective layer 56 (e.g., going from the external anatomy 60 to the internal anatomy 62) and a user 50 switches (by way of a menu or command 66 selection) from a visualization 58 of a first tissue or organ system 52a to a second tissue or organ system 52b, the second tissue or organ system 52b is provided on the visualization 58 with the user 50 and/or object 24 and/or medical instrument 34 with one or more trackers 68 engaging with (e.g., interact, move proximal toward, penetrate, manipulate, etc.) the TDPM 26.

In some embodiments, a tracker 68 includes a unique identifier (e.g., a bar code, a QR code, or other scanning code, and/or combinations thereof). Such unique identifier 68 is mapped with or attached to (e.g., physically or electronically) to an object 24 (e.g., a person, etc.) and/or medical instrument 34. In certain embodiments, the unique identifier 68 is mapped with or attached to a specific aspect of the object 24 and/or medical instrument 34 that will be engaging with (e.g., interacts with, moved proximal towards, manipulating, and/or penetrating) the TDPM 26. For instance, the unique identifier 68 is, in one embodiment, mapped with or attached to the handle of a medical instrument 34. For instance, the unique identifier 68 is, in one embodiment, mapped with or attached to the tip of the medical instrument 34 used for a procedure (e.g., providing treatment, medication, making an incision, grasping a tissue or organ, etc.). Such unique identifier 68 is scanned by the viewing device 28 and/or the interface device 38 (e.g., a computer, a scanner, a camera, an XR device). The tracker 68 (e.g., the unique identifier) is utilized to harmonize the portion of a user 50 and/or object 24 and/or medical instrument 34 including the tracker 68 with the visualization 58 and the TDPM 26. In this fashion, the portion of the user 50 and/or object 24 and/or medical instrument 34 by way of the tracker 68 is shown in the visualization 58 while engaging with (e.g., interacting, moving proximally toward, physically, manipulating, touching, etc.) the TDPM 26 are harmonized.

In further embodiments, more than one tracker 68 each including a unique identifier is associated with more than one portion or aspects of a user 50 and/or one or more objects 24 and/or one or more medical instruments 34. Accordingly, a further holistic representation can be mapped and presented in the visualization 58 as more than one mapped portion 64 of at least one user 50 and/or the object(s) 24 and/or medical instruments 34 engages with (e.g., interacts with, moves proximally towards, manipulates, penetrates, physically engages, etc.) the TDPM 26 and are harmonized.

In further embodiments, one or more trackers including a unique identifier are associated with one or more portions of the user 50 and/or one or more objects 24 and/or one or more medical instruments 34. Accordingly, one or more portions of at least one user 50 and/or one or more portions of one or more objects 24 and/or one or more portions of one or more medical instruments 34 are mapped portions 64 and presented in the visualization 58 as such one or more portions of at least one user 50 and/or one or more portions of one or more objects 24 and/or one or more portions of one or more medical instruments 34 engage with (e.g., interacts with, moves proximally towards, manipulates, penetrates, physically engages, etc.) the TDPM 26 and are harmonized.

As shown in FIG. 5, one embodiment of the present disclosure includes the harmonization of the visualization 58 (e.g. VTDO or OTDO 22) with of the TDPM 26 such that the visualization 58 is registered 42 to the correct portion of the TDPM 26. Such harmonization optionally provides a visualization 58 that operates as a visual guide for invasive procedures on internal structures (e.g., tissues, organs, etc.) of an object 24. FIG. 5 illustrates a visualization 58 harmonized with the TDPM 26 by co-locating the visualization 58 with the TDPM 26 as seen through a MICROSOFT® HOLOLENS®. As shown in exemplary FIG. 5, the external anatomical features 44 of the visualization 58 are harmonized with the external anatomy 60 of the TDPM 26. As shown in exemplary FIG. 5, the internal anatomical features 46 of the visualization 58 are harmonized with the internal anatomy 62 of the TDPM 26. The internal anatomical features 46 of the visualization 58 selected certain tissue and/or other organ systems 52 that are bones as opposed to other tissue and/or organ systems 52.

Harmonization will vary in clarity due to variations in precision, accuracy, and system limitations including the resolution of the viewing device 28, processing speeds of the controller 36 and/or the viewing device 28 and/or the interface device 38, refresh rate of the viewing device 28, internet connection speed of the viewing device 28, the interface device 38, and/or the controller 36, etc.

The one or more image data 22 regarding an object 24 (e.g., a patient, subject, etc.) are obtained by an imaging device 32. Said differently, the one or more image data 22 are obtained by an imaging device 32 capable of capturing an image of an object 24. Examples of such imaging devices 32 include those generally obtained by way of a scanning technology such as a probe (e.g., ultrasound), CT, micro-CT, MRI, where such scanning technologies can be facilitated by nuclear imaging by way of PET, and/or any other imaging capable of generating at least a 2-d image that can be combined with at least one other 2-d image to form a 3-d image. Such images are generated with the support of the aforementioned examples of medical imaging devices (“MID”) 32. The one or more image data 22 regarding a medical instrument 34 can also be generated by a scanning technology and/or by other means, including a technical drawing of the medical instrument 34, an SEM photo thereby used as a basis for a technical drawing, extrapolating/rendering a medical instrument 34 through use of indicators positioned on the medical instrument 34 such as by way of a VICON system, etc. Such technologies are also useful in monitoring movement of an object 24 (e.g., a patient or subject, medical professional, trainee, etc.), from a first position to an nth position, thereby enabling visualizations 58 such as overlays and renderings to simulate a procedure.

The system for an image guided procedure 20 includes a system controller 36. The system controller 36 is in communication with other components (e.g., the viewing device 28, the three-dimensional printing apparatus, 30, the imaging device 32, the interface device, etc.). The system controller 36 may be in communication with these components to control and/or receive signals therefrom to perform the functions described herein. The system controller 36 may include any type of computing device, computational circuit, processor(s), CPU, computer, or the like capable of executing a series of instructions that are stored in memory, including the ability to execute what is often referred to as a machine learning algorithm. The instructions may include an operating system, and/or executable software modules such as program files, system data, buffers, drivers, utilities, and the like. The executable instructions may apply to any functionality described herein to enable the inspection system to accomplish the same algorithmically and/or coordination of system components. The system controller 36 includes or is in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information, including cloud storage 70. The system controller 36 may include, or may be in communication with, an interface such as either or both of an input device that enables a user to enter data and/or instructions, and may include, or be in communication with, an interface device configured, for example to display information (e.g., a visual display that may be the same or different from the viewing device 28), or to transfer data, etc. Such interface enables a digital board, display screen, a GUI, and/or XR. Communications between the system controller 36 and other system components may be via a hardwire connection or via a wireless connection and include cloud networking/storage 70, including radio frequency, blue tooth, wi-fi, li-fi, etc. The system for an image guided procedure 20 is powered by known means and components may have separate and/or different power sources (e.g., electrical, battery, fuel cell, etc.).

Software systems compatible with the aforementioned imaging devices 32 such as MID's 32 are suitable for generating the VTDO, TDO, and/or OTDO 22 of the present disclosure. Software systems compatible with the aforementioned viewing devices 28 such as but not limited to XR devices 28 include without limitation: the INTRAVISION XR or VSI made by APOLQLAR are suitable for generating the OTDO 22 of the present disclosure. Software systems compatible with the aforementioned three-dimensional printing apparatus' 30 such as but not limited to the INTRAVISION XR are suitable for generating the OTDO 22 of the present disclosure.

Artificial intelligence by way of one or more machine learning algorithms is(are) optionally included in the software of the present disclosure.

The controller 36 is operatively connected to the imaging device 32 (e.g., MID), instructing the MID 32 by way of user inputs on an interface 38 (such as an input device such as a keyboard, mouse, stylus or GUI) to obtain at least one set of image data 22. The MID 32 transmits the at least one set of image data 22 to the controller 36 and the controller 36 thusly receives the at least one set of image data 22. The controller 36 includes software and/or algorithms capable of submitting the instructions to the MID 32 as well as receiving the instructions. The controller 36 is equipped to store and process the at least one set of image data 22, as contemplated in the present disclosure.

In some embodiments, the controller 36 is not operatively connected to the imaging device 32 (e.g., the MID) and as such, the at least one set of image data 22 is transferred to the controller 36 by way of an external drive or other forms of electronic communication such as a cloud-based 70 storage location, email, etc.

The controller 36 is operatively connected to the three-dimensional printing apparatus 30, instructing the three-dimensional printing apparatus 30 by way of user inputs on an interface 38 (such as an input device such as a keyboard, mouse, stylus or GUI). The controller 36 provides the at least one set of image data 22 to the three-dimensional printing apparatus 30 in a suitable file format such as a . OBJ, MTL (e.g., file format for color and shading generally utilized with a .OBJ file), STL, .3MF, X3D, . WLR, etc. The controller 36 transmits the at least one set of image data 22 in the aforementioned file format to the three-dimensional printing apparatus 36 and the three-dimensional printing apparatus 36 thusly receives the at least one set of image data 22 in such suitable file format. The controller 36 includes software and/or algorithms capable of submitting the instructions to the three-dimensional printing apparatus 30. The controller 36 is equipped to store and process the at least one set of image data 22, as contemplated in the present disclosure. Optionally, the three-dimensional printing apparatus 30 is equipped to store and process the at least one set of image data 22, as contemplated in the present disclosure.

In some embodiments, the controller 36 is not operatively connected to the three-dimensional printing apparatus 30 and as such, the at least one set of image data 22 is transferred to the three-dimensional printing apparatus 30 by way of an external drive or other forms of electronic communication such as a cloud-based 70 storage location, email, etc.

Referring back to FIG. 3, Once the three-dimensional printing apparatus 30 receives the at least one set of image data (or VTDO or TDO) 22, a TDPM 26 can be generated. Optionally, the user 50 can, via an interface 38, provide inputs to confirm specifications of the TDPM 26 to be generated. Optionally, the user 50 of the three-dimensional printing apparatus 30 is provided information with respect to the TDPM 26 to be generated and the three-dimensional printing apparatus 30, also by an interface 38. Suitable three-dimensional printing apparatus 30 include those that remove material from an object to generate a TDPM 26, and/or also by way of additive manufacturing. Exemplary three-dimensional printing apparatus 30 are made by STRATASYS® or similar other manufacturers, and include exemplary models such as those sold as the 8 Series such as the J850 PRO. The user 50 may select options such as one or more different colors and/or one or more different materials to further depict at least one tissue or organs 52 in the TDPM 26. Each of a first color or material is identified by reference numeral 72. Each of a second color or material is identified by reference numeral 74. One of skill in the art appreciates that there may be more than two colors and/or more than two materials utilized with a given TDPM. Such one or more materials are optionally selected based on material and mechanical properties that simulate the one or more tissues or organs 52. In some exemplary embodiments, at least two colors and/or at least two different materials are provided in the TDPM 26, where each of the at least two colors and/or at least two different materials are representative of at least two different tissues or organs 52a, 52b. Materials utilized in such three-dimensional printing apparatus 30 include various resins, metals, etc.

The physical model 26 such as a TDPM can be created to include a support structure 76 including a platform 78. The support structure 76 optionally includes a series of linkages and joints enabling the physical model 26 to be moved into different positions. The physical model 26 is attached to the support structure 76 and/or optionally is created with the physical model 26 by the three-dimensional printing apparatus 30.

The controller 36 is operatively connected to the viewing device 28, instructing the viewing device 28 by way of user 50 inputs on an interface 38 (such as an input device that includes but is not limited to: a keyboard, mouse, stylus, GUI, etc.). The controller 36 provides the at least one set of image data 22 to the viewing device 28 in a suitable file format such as a .OBJ, .MTL (e.g., file format for color and shading generally utilized with a .OBJ file), .STL, .3MF, .X3D, .WLR, etc. The controller 36 transmits the at least one set of image data (or VTDO) 22 in the aforementioned file format to the viewing device 28 and the viewing device 28 thusly receives the at least one set of image data (or VTDO) 22 in such suitable file format. The controller 36 includes software and/or algorithms capable of submitting the instructions to the viewing device 28. The controller 36 is equipped to store and process the at least one set of image data (or VTDO) 22, as contemplated in the present disclosure. Optionally, the viewing device 28 is equipped to store and process the at least one set of image data (or VTDO) 22, as contemplated in the present disclosure.

In some embodiments, the controller 36 is not operatively connected to the viewing device 28 and as such, the at least one set of image data (or VTDO) 22 is transferred to the viewing device 28 by way of an external drive or other forms of electronic communication such as a cloud-based 70 storage location, email, etc.

In certain embodiments, utilizing a viewing device 28, controller 36, and interface device 38, with specific properties is desirous, particularly if relating to a specific type of medical procedure (as will be discussed in greater detail below). For instance, one or more of the following properties may be preferable: resolution, processing speed, random access memory (RAM), internet connection speed, upload rate, download rate, internet signal strength, etc. In some embodiments, the viewing device 28, controller 36, and interface device 38, has 2 gigabytes of RAM, 4 gigabytes of RAM, 8 gigabytes of RAM, 16 gigabytes of RAM, 24 gigabytes of RAM, 32 gigabytes of RAM, 48 gigabytes of RAM, or between 2 gigabytes and 48 gigabytes of RAM. In some embodiments, the viewing device 28, controller 36, and interface device 38, has a refresh rate of at least 60 HZ, at least 120 HZ, or at least 240 HZ, or between 60 HZ and 240 HZ. In some embodiments, the viewing device 28 includes a minimum resolution of 780P, 1080P, 2140P, 4280P, etc. In some embodiments, the viewing device 28 is at least one XR device. In some such embodiments, the viewing device 28 is at least one VR, AR and/or MR device.

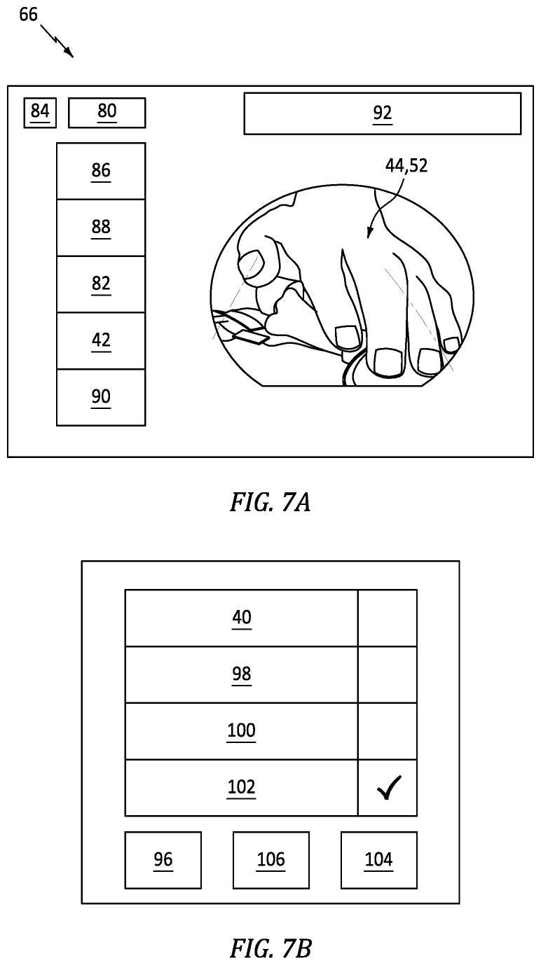

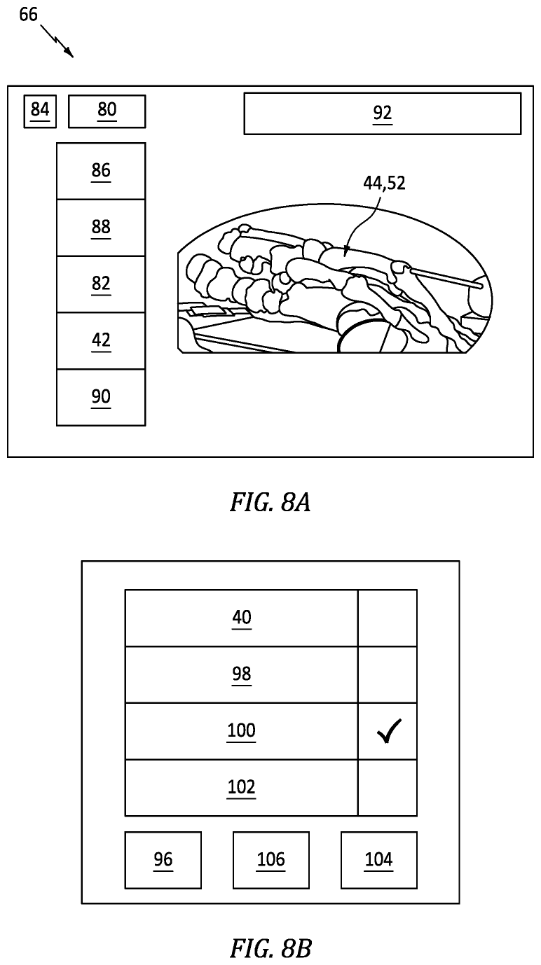

As exemplified in FIGS. 6A-7B, various menu options 66 are provided on the visualization 58 by way of the XR device which is both an interface device 38 and a viewing device 28. Focusing on exemplary FIGS. 7A and 8A, menu options 66 such as the main menu 80, home 82, back 84, save (and optionally, save offline) 86, segmentation 40, cutting plane 88, boundary box (or registration) 42, and information (or settings, support, profile, etc.) 90 are provided. In greater detail, the cutting plane option enables a user to view only a portion of the visualization 58 (e.g., OTDO 22). By way of example, the visualization 58 (e.g., OTDO 22) may include the entire torso of a patient or subject 24, and the user can choose to limit the visualization 58 to only a portion of the torso. The boundary box (or registration) 42 allows a user to harmonize the visualization 58 to the TDPM 26. The boundary box (or registration) 42 includes nodes 94 that allow the user to align the visualization 58 to the TDPM 26. Optionally, branding 92 is provided on the screen.

Focusing on exemplary FIGS. 7B and 8B, upon selecting the segmentation 40 option, additional menu options are provided. Segmentation 40 options such as kwire 96, fiducials 98, bones 100, and soft tissue 102 allow the user to select certain of tissues or organs 52 that will be a part of the visualization 58. Optionally a “select all” 106 option is available enabling a user to segment 40 with multiple tissue or organ groups 52, and/or also with a wire frame 96 such as a surface feature format and/or the kwire format. One of skill in the art appreciates that other menu options are possible. With regard to FIGS. 7A and 7B, the menu option for soft tissue 102 was selected by the user 50. With regard to FIGS. 8A and 8B, the menu option for bones 100 was selected by the user 50. Referring back to FIG. 6a, the menu option for soft tissue 102 was selected by the user 50. Referring back to FIGS. 6B and 6C, the menu option for wireframe (e, g., kwire) 96 was selected by the user 50.

Further menu options enable recording and saving, and thereafter playing back the recording. During such a recording, additional features such as rewind 108, play 110, and or fast forward 112 are provided. Such features allow for rewinding or fast-forwarding the recording at a specific time interval (e.g. 10 seconds, 20 seconds, 30 seconds, etc.), and an exemplary time interval bar (running horizontally across within the lower third of the visualization) can be seen on FIGS. 6A-6C, where FIG. 6B shows the video has elapsed by way of the progress circle on the time interval bar. The play 110 button optionally functions as a stop or pause button also.

While the present disclosure exemplifies menus 66 in various locations such as on the left-hand side, top, and bottom, menus 66 and prompts can be provided in other locations, including the center and/or the right-hand side. Similarly, the order of menu options 66 can vary depending on the software and/or user preferences. Similarly, other synonyms, acronyms or abbreviations may be utilized for names of menu options 66.

The present disclosure is useful in a number of in vitro and in vivo procedures, including those delivering medical care to a patient, training medical professionals, performing research, etc. The present disclosure, by way of the visualization 58 viewable by way of the at least one viewing device 28 (e.g., more than one viewing device 28 such that multiple medical personnel and/or students and/or patients can engage with and/or simply view the visualization 58, and others can engage both with a viewing device 28 harmonized with the TDPM 26) and controller 36, and optionally by way of the at least one TDPM 26 (e.g., more than one TDPM 26 that are replicates and/or are variants), and further includes activities such as virtual or remote consultation, virtual or remote mentoring, medical procedure planning, medical procedure practice, and also guidance during a medical procedure. Such activities may include multiple users utilizing computers, mobile devices, tablets, etc., with headsets, and/or other scenarios where multiple users having XR viewing devices 28 such as XR headsets and are thusly able to engage with the visualization 58. In some such embodiments, certain users may have “view only” ability or further limited functionality such that only their local visualization 58 is modified, while other users may have unlimited ability and are thus able to modify functionality of the visualization 58 for more than one user.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a” “an” and “the” refer to one or more than one, unless the context clearly dictates otherwise. For example, the term “comprising a specimen” includes single or plural specimens and is considered equivalent to the phrase “comprising at least one specimen”. The term “o” refers to a single element of stated alternative elements or a combination of two or more elements unless the context clearly indicates otherwise. As used herein, “comprise” means “includes”. Thus, “comprising A or B” means “including A or B, or A and B” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. § 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosure-such as alternative materials, structures, configurations, methods, devices, and components, and so o-may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

What is claimed is:

1. A system for visually guided in-vitro procedures, comprising:
one or more image data having a file format, the one or more image data including a first tissue or organ and a second tissue or organ that is different from the first tissue or organ;
one or more physical models; and
one or more viewing devices;
wherein the one or more image data is configured to provide a VTDO including the first tissue or organ and the second tissue or organ, wherein the VTDO is configured to provide a visualization viewable on the one or more viewing devices;
wherein the one or more image data is utilized to generate the one or more physical models comprising a TDPM, the TDPM including the first tissue or organ and the second tissue or organ;
wherein the visualization, when viewed through the one or more viewing devices comprising an XR device and harmonized with the TDPM synchronizes the first tissue or organ and the second tissue or organ both through the visualization and physically on the TDPM.

2. The system for visually guided in-vitro procedures according to claim 1, wherein the one or more viewing devices further comprises a screen.

3. The system for visually guided in-vitro procedures according to claim 1, wherein a user of the XR device is able to synchronize the visualization and the TDPM as the user interacts with at least the TDPM in real time.

4. The system for visually guided in-vitro procedures according to claim 2, wherein a user of the XR device is able to harmonize the visualization and the TDPM as the user interacts with both the TDPM and the visualization in real time.

5. The system for visually guided in-vitro procedures according to claim 1, further comprising an in vivo patient or subject.

6. The system for visually guided in-vitro procedures according to claim 5, wherein the image data corresponds to at least a portion of the in vivo patient or subject.

7. The system for visually guided in-vitro procedures according to claim 6, wherein the VDO is configured to provide an OTDO, wherein the OTDO is configured by the XR device to generate a visualization through the XR device that is harmonized with the TDPM, wherein the visualization through the XR device is further harmonized with the portion of the in vivo patient or subject that corresponds to the image data.

8. The system for visually guided in-vitro procedures according to claim 1, wherein the first tissue or organ is an external tissue or organ that is viewable to the naked eye, and the second tissue or organ is an internal tissue or organ that is obfuscated.

9. The system for visually guided in-vitro procedures according to claim 1, further comprising a third tissue or organ that is different from the first tissue or organ and the second tissue or organ.

10. The system for visually guided in-vitro procedures according to claim 1, wherein the XR device is an MR device.

11. The system for visually guided in-vitro procedures according to claim 1, wherein the one or more image data are generated by an MID.

12. The system for visually guided in-vitro procedures according to claim 1, wherein the one or more image data undergoes at least one of segmentation and registration.

13. The system for visually guided in-vitro procedures according to claim 1, wherein the TDPM includes at least two materials, wherein each of the at least two materials is either a different color and/or has different material or mechanical properties.

14. A method for a visually guided procedure, the method comprising:
obtaining a VTDO that includes a first tissue or organ and a second tissue or organ;
creating a TDO from the VTDO;
creating a TDPM from the TDO, the TDPM having the first tissue or organ and the second tissue or organ;
manifesting a visualization configured from the VTDO through an interface device;
registering an OTDO in order to harmonize the visualization through a viewing device with a physical model;
wherein the visualization of the first tissue or organ and the second tissue or organ through the viewing device synchronizes with the TDPM such that a user is able to interact virtually through the viewing device with the visualization and physically with the TDPM;
wherein the interface device and the viewing device comprise an XR device.

15. The method for the visually guided procedure according to claim 14, wherein the viewing device further comprises a screen.

16-30. (canceled
